(19) United States
(12) Patent Application Publication
(10) Pub. No.: US 2013/0060087 A1
(43) Pub. Date: Mar. 7, 2013

(54) RIGID SCOPE APPARATUS

(71) Applicant: Koji Yoshida, Kanagawa-ken (JP)

(72) Inventor: Koji Yoshida, Kanagawa-ken (JP)

(73) Assignee: FUJIFILM Corporation, Tokyo (JP)

(21) Appl. No.: 13/485,695

(22) Filed: May 31, 2012

(30) Foreign Application Priority Data
Sep. 6, 2011 (JP) 128926/2011

(51) Int. Cl. A61B 1/05; A61B 1/06

(52) U.S. Cl. 600/112

(57) ABSTRACT

A rigid scope apparatus including a rigid scope body to be inserted into a body for guiding and projecting an inputted illumination light on an examination area of the body, and receiving and guiding a light from the examination area caused by projection of the illumination light, and a camera head section to be connected to the rigid scope body and having an imaging section for receiving the light from the examination area and guided by the rigid scope body, and outputting an image signal. The camera head section includes an illumination light output section for outputting the illumination light and the rigid scope body receives and guides the illumination light outputted from the illumination light output section.

### BACKGROUND OF THE INVENTION

1. Field of the Invention

The present invention relates to a rigid scope apparatus having a rigid scope body to be inserted into a human body for projecting an illumination light on an examination area inside of the body and guiding an image of the examination area produced by the projection of the illumination light, and a camera head section for receiving the image guided by the rigid scope body and outputting an image signal.

2. Description of the Related Art

Endoscope systems for examining tissues of body cavities are known widely, and endoscope systems for capturing an ordinary image of an examination area in a body cavity by projecting a white light on the examination area and displaying the captured ordinary image on a monitor screen are used widely.

As one of such endoscope systems, a system that uses a so-called rigid scope in which an image of examination area is guided by a relay lens is proposed.

More specifically, a system in which a camera head section having an image sensor is connected to the eyepiece section of a rigid scope body having a relay lens and an image of examination area guided by the relay lens of the rigid scope body is inputted to the camera head section via the eyepiece section to form the image on the image sensor of the camera head section, thereby obtaining an image signal, is proposed as described, for example, in Japanese Unexamined Patent Publication No. 10 (1998)-290780.

Here, in an endoscope system using the rigid scope described above, the white light is guided by the rigid scope body and projected on an examination area from the tip thereof, and it is generally of a configuration in which a light guide is connected to the rigid scope body via a connector to guide a white light emitted from the light source to the rigid scope body and the white light is projected on the examination area therefrom, as in the rigid scope system described in Japanese Unexamined Patent Publication No. 10 (1998)-290780.

In the rigid scope system described above, a signal cable for outputting an image signal is connected to the camera head section in addition to the light guide connected to the rigid scope body. If the system has such configuration in which the light guide and signal cable are connected separately, the overall size of the rigid scope system, including these connection sections, rigid scope body, and camera head section, becomes large and the operability is degraded when used in a surgical operation or the like. In addition, when moving the rigid scope system, the movement is restricted by the lengths of the light guide and the signal cable. Thus, from this viewpoint also, such system provides poor operability and the doctor may feel stressed during the surgical operation.

Further, separate connections of the light guide and signal cable to the rigid scope body and camera head section respectively requires time.

In view of the circumstances described above, it is an object of the present invention to provide a rigid scope apparatus having a rigid scope body and a camera head section to be connected to the rigid scope body in which overall size reduction is realized with improved operability.

### SUMMARY OF THE INVENTION

A rigid scope apparatus of the present invention is an apparatus, including:

a rigid scope body to be inserted into a body for guiding and projecting an inputted illumination light on an examination area of the body, and receiving and guiding a light from the examination area caused by the projection of the illumination light; and

a camera head section to be connected to the rigid scope body and having an imaging section for receiving the light from the examination area and guided by the rigid scope body, and outputting an image signal, wherein:

the camera head section includes an illumination light output section for outputting the illumination light; and

the rigid scope body receives and guides the illumination light outputted from the illumination light output section.

In the rigid scope apparatus of the present invention, the illumination light output section may include an optical fiber for guiding and outputting the illumination light, or a phosphor that emits the illumination light and an optical fiber for guiding an excitation light to be projected on the phosphor.

Further, the apparatus may include a rigid scope connection detection section for detecting as to whether or not the rigid scope body is connected to the camera head section, and the illumination light output section may be a section that outputs the illumination light while connection between the rigid scope body and the camera head section is detected by the rigid scope connection detection section and does not output the illumination light while connection between the rigid scope body and the camera head section is not detected.

Still further, the apparatus may include a camera head connection detection section for detecting as to whether or not the camera head section is connected to a light source apparatus that emits a light to be inputted to the camera head section, and the illumination light output section may be a section that outputs the illumination light while connection between the rigid scope body and the camera head section is detected by the rigid scope connection detection section and connection between the camera head section and the light source apparatus is detected by the camera head connection detection section, and does not output the illumination light while connection between the rigid scope body and the camera head section or connection between the camera head section and the light source apparatus is not detected.

Further, at least one of the rigid scope body and the camera head section may include a light blocking section for preventing the illumination light outputted from the illumination light output section from entering into the imaging section of the camera head section.

Still further, the illumination light output section may be a section that outputs a plurality of illumination lights having different wavelength ranges.

Further, one of the plurality of illumination lights may be a white light.

Still further, one of the plurality of illumination lights may be a near infrared light or an autofluorescence excitation light for exciting autofluorescence in the examination area.

Further, the rigid scope body may includes a light receiving section for receiving a light having the plurality of illumination lights mixed therein.

Still further, the rigid scope body may include a plurality of specific wavelength light receiving sections for receiving the plurality of illumination lights separately.

Further, each of the plurality of specific wavelength light receiving sections may be provided at a different radial position from a center position of a connection face of the rigid scope body with the camera head section.

Still further, the plurality of specific wavelength light receiving sections may be provided concentrically with respect to the center position described above.

Further, the illumination light output section may include a diffusion section for diffusing the illumination light.

Still further, the apparatus may include a transmission member between an output face of the illumination light output section and a light receiving face of the rigid scope body for receiving the illumination light, the transmission member having a refractive index substantially identical to a refractive index adjacent to the output face or a refractive index adjacent to the light receiving face, and transmitting the illumination light outputted from the illumination light output section.

Further, the transmission member may be a member having elasticity.

Still further, the transmission member may be a member formed of a resin.

Further, the transmission member may be detachably attachable to the camera head section or the rigid scope body.

Still further, the apparatus may include a supply member for supplying a liquid or gel between an output face of the illumination light output section for outputting the illumination light and a light receiving face of the rigid scope body for receiving the illumination light, the liquid or gel having a refractive index substantially identical to a refractive index adjacent to the output face or a refractive index adjacent to the light receiving face.

Further, the illumination light output section may be provided in a peripheral portion of the imaging section.

The rigid scope apparatus of the present invention includes a rigid scope body and a camera head section in which the camera head section includes an illumination light output section for outputting an illumination light, and the rigid scope body receives and guides the illumination light outputted from the illumination light output section, thereby projecting the illumination light on an examination area. This eliminates the need to connect a light guide to the rigid scope body, as required in the conventional rigid scope apparatus, resulting in an overall downsizing of the apparatus and operability improvement by saving the effort of connecting and maneuvering the light guide.

In the rigid scope apparatus of the present invention, in the case where an optical fiber for guiding and outputting the illumination light is provided in the camera head section, as the illumination light output section, an existing light source apparatus can be used, that is, all that is required is to simply connect the light source apparatus to the camera head section, so that the rigid scope apparatus of the present invention may be realized inexpensively with a simple structure.

Further, in the case where a phosphor that emits the illumination light and an optical fiber for guiding an excitation light to be projected on the phosphor are provided in the camera head section, as the illumination light output section, the tip of the rigid scope body may be thinned and optimized with freedom in the shape thereof (direct viewing, oblique viewing, and the like) in comparison, for example, with the case where the phosphor described above is provided at the tip of the rigid scope body.

Still further, in the case where the apparatus includes a rigid scope connection detection section for detecting as to whether or not the rigid scope body is connected to the camera head section, and the illumination light output section is a section that outputs the illumination light while connection between the rigid scope body and the camera head section is detected by the rigid scope connection detection section and does not output the illumination light while connection between the rigid scope body and the camera head section is not detected, a high intensity light is prevented from being accidentally outputted from the camera head section before the rigid scope body is connected to the camera head section, thus the high intensity light is prevented from being accidentally incident on the human eyes, leading to safety improvement.

Further, in the case where the apparatus further includes a camera head connection detection section for detecting as to whether or not the camera head section is connected to a light source apparatus that emits a light to be inputted to the camera head section, and the illumination light output section is a section that outputs the illumination light while connection between the rigid scope body and the camera head section is detected by the rigid scope connection detection section and connection between the camera head section and the light source apparatus is detected by the camera head connection detection section, and does not output the illumination light while connection between the rigid scope body and the camera head section or connection between the camera head section and the light source apparatus is not detected, a high intensity light is prevented from being accidentally outputted from the camera head section or from the light source apparatus before the rigid scope body is connected to the camera head section or before the camera head section is connected to the light source apparatus, thus the high intensity light is prevented from being accidentally incident on the human eyes, leading to safety improvement.

Still further, in the case where at least one of the rigid scope body and the camera head section includes a light blocking section for preventing the illumination light outputted from the illumination light output section from entering into the imaging section of the camera head section, noise generation in the image signal due to entrance of the illumination light into the imaging section may be prevented.

Further, in the case where the rigid scope body includes a plurality of specific wavelength light receiving sections for receiving the plurality of illumination lights separately, each specific wavelength light receiving section may be formed of a material most suitable for the wavelength characteristics of the illumination light to be received thereby, so that the transmittance of the illumination light may be improved.

Still further, in the case where each illumination light is outputted from the camera head section at a different radial position from a center position of a connection face of camera head section with the rigid scope body, and a plurality of specific wavelength light receiving sections is provided concentrically with respect to the center position, each illumination light outputted from the camera head section may be reliably received by the corresponding specific wavelength light receiving section even when the rigid scope body and the camera head section are relatively rotated at the connection face.

Further, in the case where the illumination light output section of the camera head section includes a diffusion section for diffusing the illumination light, then, for example, even when a laser light is used as the illumination light, the laser light may be diffused by the diffusion section, whereby a high intensity laser light may be prevented from being outputted from the camera head section and incident on the human eyes, leading to safety improvement.

Still further, in the case where the apparatus includes a transmission member between an output face of the illumination light output section and a light receiving face of the rigid scope body for receiving the illumination light, the transmission member having a refractive index substantially identical to a refractive index adjacent to the output face or a refractive index adjacent to the light receiving face, and transmitting the illumination light outputted from the illumination light output section, or the apparatus includes a supply member for supplying a liquid or gel between an output face of the illumination light output section for outputting the illumination light and a light receiving face of the rigid scope body for receiving the illumination light, the liquid or gel having a refractive index substantially identical to a refractive index adjacent to the output face or a refractive index adjacent to the light receiving face, degradation in light guiding efficiency that may occur in a spatial optical connection between the output face of the illumination light output section and light receiving face of the rigid scope body may be prevented and high light guiding efficiency may be ensured.

### BRIEF DESCRIPTION OF THE DRAWINGS

FIG. 1 is a schematic configuration diagram of a rigid scope system that employs an embodiment of a rigid scope apparatus of the present invention.

FIG. 2 schematically illustrates a configuration of the rigid scope body and camera head section.

FIG. 3 is a diagram of the rigid scope body viewed from the arrow “A” direction shown in FIG. 2.

FIG. 4 is a longitudinal cross-sectional view of the rigid scope body.

FIG. 5 is a diagram of the camera head section viewed from the arrow “B” direction shown in FIG. 2.

FIG. 6 is a schematic view of the camera head section, illustrating the internal configuration thereof.

FIG. 7 illustrates, byway of example, a spectrum of light emitted from the white light output section.

FIG. 8 is a block diagram of a processor and light source apparatus, schematically illustrating the configuration thereof.

FIG. 9 illustrates an embodiment in which a rigid scope connection detection section and a camera head section connection detection section are provided.

FIG. 10 illustrates an embodiment in which a diffusion section is provided on the camera head section.

FIG. 11 illustrates an embodiment in which a transmission member is provided between an output face of the camera head section and an incident face of the rigid scope body.

FIG. 12 illustrates an embodiment in which a supply member for supplying matching oil is provided between the output face of the camera head section and the input face of the rigid scope body.

FIG. 13A illustrates an embodiment in which the white light projection section and near infrared light projection section are provided at different radial positions.

FIG. 13B illustrates an embodiment in which a fiber bundle for white light and a fiber bundle for near infrared light are disposed concentrically.

### DESCRIPTION OF THE PREFERRED EMBODIMENTS

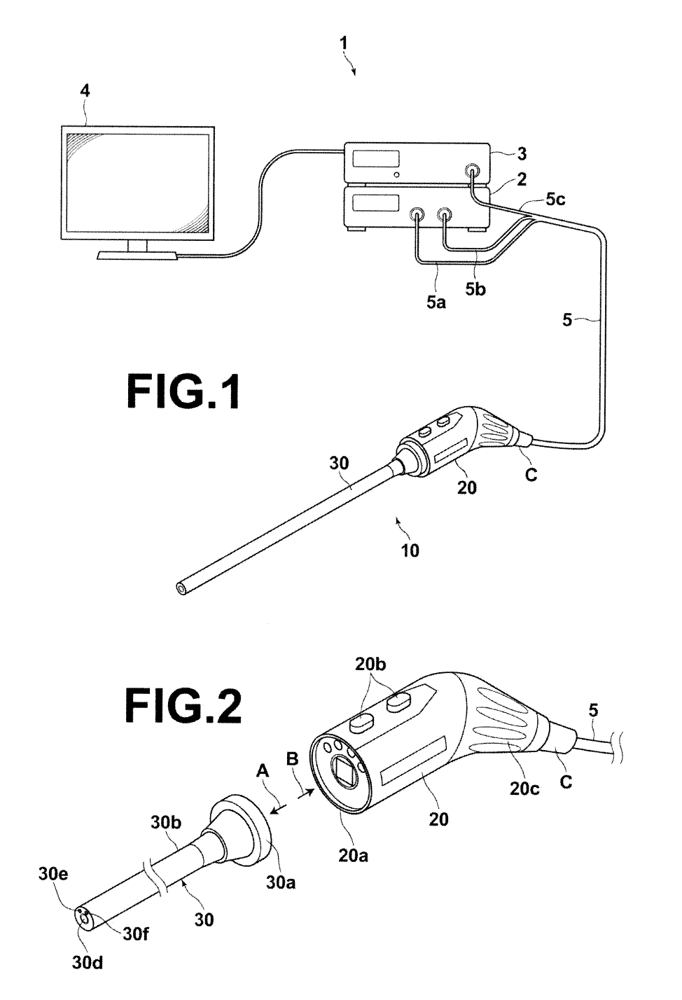

Hereinafter, a rigid scope system that employs an embodiment of the rigid scope apparatus of the present invention will be described with reference to the accompanying drawings. FIG. 1 is an external view of a rigid scope system 1 according to the present embodiment, illustrating the schematic configuration thereof.

As illustrated in FIG. 1, the rigid scope system 1 of the present embodiment includes a light source apparatus 2 for emitting an excitation light which is a blue light for exciting a phosphor, to be described later, and a near infrared light for exciting ICG, a rigid scope imaging apparatus 10 for guiding and projecting a white light, emitted from the phosphor by the projection of the excitation light, and the near infrared light on an examination area, and imaging an ordinary image based on a reflection light reflected from the examination area by the projection of the white light and a fluorescence image based on fluorescence emitted from the ICG of the examination area by the projection of the near infrared light, a processor 3 for performing predetermined processing on image signals captured by the rigid scope imaging apparatus 10, and monitor 4 for displaying an ordinary image and a fluorescence image of the examination area based on a display control signal generated by the processor 3.

As illustrated in FIG. 1, a camera head section 20 of the rigid scope imaging apparatus 10 is connected to the light source apparatus 2 and a processor 3 via a cable 5. The cable 5 is a bundle of three cables, an excitation light optical cable 5a for guiding the excitation light emitted from the light source apparatus 2, a near infrared light optical cable 5b for guiding the near infrared light emitted from the light source apparatus 2, and a signal cable 5c for transmitting an image signal outputted from the camera head section 20, and is connected to the camera head section 20 via a connector C integrally accommodating these cables. Note that the near infrared light and excitation light may be combined using a lens, mirror, or the like inside of the light source apparatus 2 and in which case the number of cables connected to the light source apparatus 2 may be reduced to one.

The cable 5 may be detachably attachable to the camera head section 20 via the connector C or fixedly attached to the camera head section 20. Further, the cable 5 is detachably attachable to the light source apparatus 2 and processor 3.

As illustrated in FIG. 1, the rigid scope imaging apparatus 10 includes a rigid scope body 30 to be inserted into a body cavity of a patient and the camera head section 20 for capturing an ordinary image and a fluorescence image of an examination area guided by the rigid scope body 30. As illustrated in FIG. 2, the rigid scope imaging apparatus 10 is constituted by the rigid scope body 30 and camera head section 20 detachably attached to each other. The mechanism for detachably attaching the rigid scope body 30 to the camera head section 20 may be, for example, a screw mechanism or any other known mechanism.

The rigid scope body 30 includes a connection section 30a, insertion member 30b, an imaging window 30d, and light projection windows 30e, 30f.

The connection section 30a is provided at one end of the rigid scope body 30 (insertion member 30b) on the side of the camera head section 20, and is to be detachably attached to the connection section of the camera head section 20, as described above.

The insertion member 30b is a member to be inserted into a body cavity when imaging of the body cavity is performed. The insertion member 30b is formed of a rigid material and has, for example, a cylindrical shape with a diameter of about 5 mm.

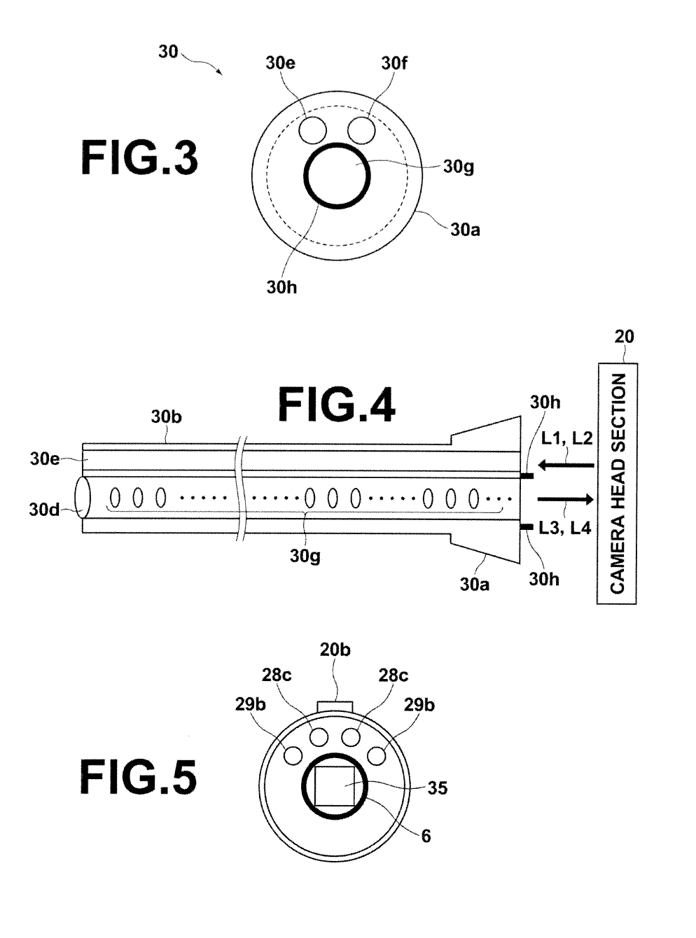

FIG. 3 is a diagram of the connection section 30a of the rigid scope body 30 shown in FIG. 2 viewed from the arrow “A” direction, and FIG. 4 is a longitudinal cross-sectional view of the rigid scope body 30 shown in FIG. 2.

As shown in FIGS. 3 and 4, the rigid scope body 30 includes therein two fiber bundles 30e, 30f. The fiber bundles 30e, 30f are bundled multimode fibers and extended from one end of the rigid scope body 30 on the side of the connection section 30a to a distal end on the opposite side. The tip portions of the fiber bundles 30e, 30f are polished to form the light projection windows, and the white light and near infrared light are projected from the respective windows onto the examination area. In the present embodiment, it is assumed that the white light and near infrared light are mixed and inputted to each of the fiber bundles 30e, 30f, and guided thereby. Note that a light receiving portion of the fiber bundle 30e or 30f corresponds to the light receiving section in the claims. Further, in the present embodiment, two fiber bundles 30e, 30f are provided, but only one or more than two fiber bundles may be provided. Further, the cross-sectional shape of the fiber bundle is not limited to a circular shape as shown in FIG. 3, and may be an arc shape, such as a crescent shape.

Further, the rigid scope body 30 includes therein a relay lens 30g for forming an ordinary image L3 and fluorescence image L4 of the examination area inputted from the imaging window 30d, and guiding the images to and outputting from the end portion of the rigid scope body 30 on the side of the camera head section 20, as shown in FIGS. 3 and 4. The ordinary image L3 and fluorescence image L4 outputted from the relay lens 30g are inputted to the camera head section 20.

As illustrated in FIGS. 3 and 4, a light blocking section 30h formed in a circle around the relay lens 30g is provided on the surface of the connection section 30a of the rigid scope body 30 on the side of the camera head section 20. The light blocking section 30h is provided to prevent the white light and near infrared light outputted from the camera head section 20 from entering into the relay lens 30g and an imaging unit 35 of the camera head section 20, to be described later, when the rigid scope body 30 is connected to the camera unit 20. As for the material of the light blocking section 30h, for example, an elastic body, such as a colored resin, may be used.

The camera head section 20 includes an imaging unit 35 for capturing ordinary image L3 and fluorescence image L4 outputted from the relay lens 30g of the rigid scope body 30. Further, the camera head section 20 includes a connection section 20a formed on the side of the rigid scope body 30 to be detachably attached to the rigid scope body 30 and a handle section 20c formed on the side of connector C where cable 5 is connected in order to facilitate the user to hold the camera head section 20. Further, operation buttons 20b are provided on the camera head section 20. The operation buttons 20b are provided for receiving a user instruction, such as an instruction to project the white light and near infrared light. Further, although not shown in the present embodiment, if a discharge hole is provided for discharging a liquid or gas, the operation buttons 20b may also receive an instruction to eject a liquid or gas from the discharge hole.

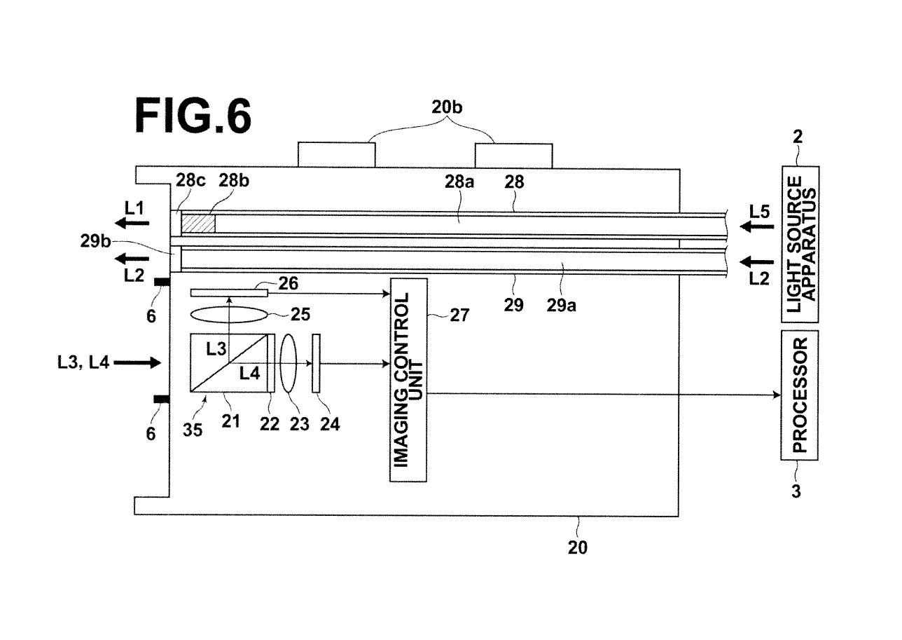

FIG. 5 is a diagram of the camera head section 20 viewed from the arrow “B” direction, and FIG. 6 is a schematic view of the camera head section 20, illustrating the internal configuration thereof.

The camera head section 20 includes an imaging unit 35 having a first imaging system that captures a fluorescence image L4 of an examination area formed by the relay lens 30g in the rigid scope body 30 and generates a fluorescence image signal of the examination area, and a second imaging system that captures an ordinary image L3 of the examination area formed by the relay lens 30 in the rigid scope body 30 and generates an ordinary image signal. These imaging systems are separated into two orthogonal optical axes by a dichroic prism 21 having a spectral characteristic that reflects the ordinary image L3 and transmits the fluorescence image L4.

The first imaging system includes an excitation light cut filter 22 that cuts lights having wavelengths not greater than that of the excitation light reflected from the examination area and transmitted through the dichroic prism 21, and transmits a fluorescence wavelength range illumination light to be described later, a first image forming optical system 23 that forms the fluorescence image L4 outputted from the rigid scope body 30 and transmitted through the dichroic prism 21 and excitation light cut filter 22, and a high sensitivity image sensor 24 that captures the fluorescence image L4 formed by the first image forming optical system 23.

The high sensitivity image sensor 24 detects a light in a wavelength range of the fluorescence image L4 with high sensitivity, converts the detected light to a fluorescence image signal, and outputs the fluorescence image signal. As for the high sensitivity image sensor 24, a monochrome image sensor may be used.

The second imaging system includes a second image forming optical system 25 that forms the ordinary image L3 outputted from the rigid scope body 30 and reflected from the dichroic prism 21, and an image sensor 26 that captures the ordinary image L3 formed by the second image forming optical system 25.

The image sensor 26 detects a light in the wavelength range of ordinary image L3, converts the detected light to an ordinary image signal, and outputs the image signal. Color filters of three primary colors, red (R), green (G), and blue (B) or color filters of cyan (C), magenta (M), and yellow (Y) are arranged on the imaging surface of image sensor 26 in a Beyer or honeycomb pattern.

The camera head section 20 includes an imaging control unit 27. The imaging control unit 27 performs CDS/AGC (correlated double sampling/automatic gain control) and A/D conversion on a fluorescence image signal outputted from the high sensitivity image sensor 24 and an ordinary image signal outputted from the image sensor 26, and outputs resultant image signals to the processor 3 through the cable 5 (FIG. 1).

As illustrated in FIGS. 5 and 6, a white light output section 28 that guides an excitation light outputted from the light source apparatus 2, emits a white light based on the excitation light, and inputs the emitted white light to the fiber bundles 30e, 30f of the rigid scope body 30, and a near infrared output section 29 that guides a near infrared light outputted from the light source apparatus 2 and inputs the near infrared light to the fiber bundles 30e, 30f of the rigid scope body 30 are provided adjacent to the imaging unit 35 inside of the camera head section 20. The white light output section 28 and the near infrared light output section 29 correspond to the illumination light output section.

The white light output section 28 includes an excitation light multimode optical fiber 28a for guiding the excitation light outputted from the light source apparatus 2, a phosphor 28b which is excited by absorbing a part of the excitation light (blue light) guided by the excitation light multimode optical fiber 28a to emit a green to yellow visible light, and a light projection window 28c for projecting the white light emitted from the phosphor 28b on input faces of the fiber bundles 30e, 30f. The phosphor 28b is made of a plurality of types of fluorescence materials and includes, for example, a YAG system phosphor or a fluorescence material, such as BAM (BaMgAl₁₀O₁₇) or the like.

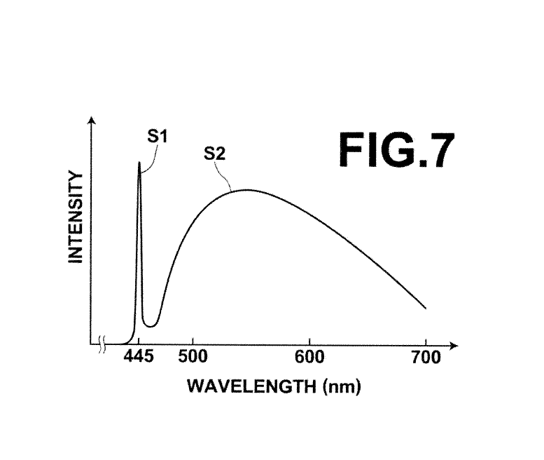

FIG. 7 illustrates, by way of example, a spectrum of the light emitted from the white light output section 28. As shown in FIG. 7, a light having a blue light spectrum S1 that transmits through the phosphor 28b and a light having a green to yellow visible light spectrum 32 are outputted from the white light output section 28.

The term “white light” as used herein is not strictly limited to a light having all wavelength components of visible light and may include any light as long as it includes light in a specific wavelength range, for example, primary light of R (red), G (green), or B (blue). Thus, in a broad sense, the white light may include, for example, lights having wavelength components from green to red, lights having wavelength components from blue to green, and the like. Although the white light output section 28 outputs the blue light spectrum S1 and visible light spectrum S2 shown in FIG. 7, the light of these spectra is also regarded as the white light.

The near infrared light output section 29 includes a near infrared light multimode optical fiber 29a for guiding the near infrared light outputted from the light source apparatus 2 and a light projection window 29b for projecting the near infrared light guided by the near infrared light multimode optical fiber 29a on input faces of the fiber bundles 30e, 30f.

As for the multimode optical fiber used in the white light output section 28 and near infrared output section 29, for example, a small diameter optical fiber having a diameter of 0.3 mm to 0.5 mm, including an outer protection coating layer, with a core diameter of 105 μm and a clad diameter of 125 μm may be used.

FIG. 6 illustrates one white light output section 28 and one near infrared light output section 29 but, in fact, two white light output sections 28 and two near infrared light output sections 29 are provided, as illustrated in FIG. 5. As illustrated in FIG. 5, each near infrared light output section 29 is disposed on each side of the white light output sections 28 disposed adjacent to each other.

Further, it is desirable that a structure for placing the optical axes of the white light output sections 28 and near infrared light output sections 29 in the camera head section 20 and optical axes of the fiber bundles 30e, 30f in the rigid scope body 30 adjacent to each other be provided. For example, it is preferable that a structure for restricting the rotation of the rigid scope body 30 in a circumferential direction with respect to the connection section 20a of the camera head section 20 be provided on each of the connection section 20a of the camera head section 20 and the connection section 30a of the rigid scope body 30. More specifically, the rotation of the rigid scope body 30 in a circumferential direction with respect to the connection section 20a of the camera head section 20 may be restricted by connecting the connection section 20a of the camera head section 20 and the connection section 30a of the rigid scope body 30 by the screw mechanism described above or by providing a protrusion, for example, on the connection section 30a of the rigid scope body 30 and a recess that fits the protrusion in the connection section 20a of the camera head section 20.

As illustrated in FIGS. 5 and 6, the light blocking section 6 formed in a circle around the imaging unit 35 is provided on the surface of the connection section 20a of the camera head section 20 on the side of the rigid scope body 30. The light blocking section 6 is provided to prevent the white light and near infrared light outputted from the camera head section 20 from entering into the relay lens 30g and imaging unit 35 of the camera head section 20 when the rigid scope body 30 is connected to the camera unit 20. As for the material of the light blocking section 6, for example, an elastic body, such as a colored resin, may be used. In the present embodiment, the light blocking section 30h is provided on the rigid scope body 30, but it may be provided on the camera head section 20.

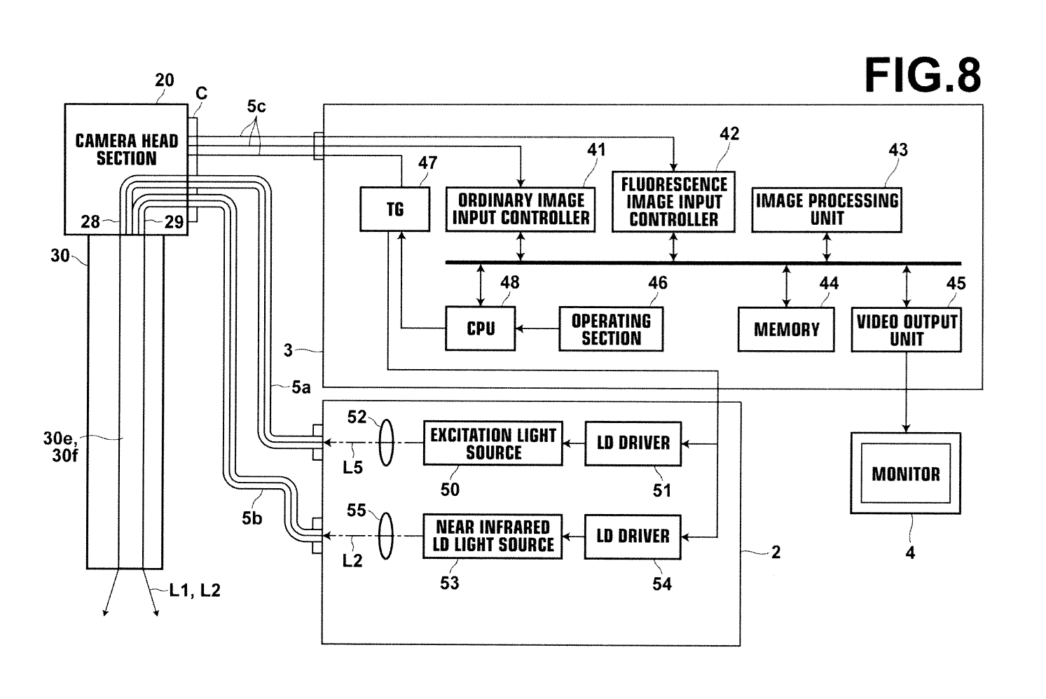

As illustrated in FIG. 8, the processor 3 includes an ordinary image input controller 41, a fluorescence image input controller 42, image processing unit 43, memory 44, video output unit 45, operating section 46, TG (timing generator) 47, and CPU 48.

Each of the ordinary image input controller 41 and fluorescence image input controller 42 is provided with a line buffer having a predetermined capacity. The ordinary image input controller 41 temporarily stores ordinary image signals with respect to each frame outputted from the imaging control unit 27 of camera head section 20 and the fluorescence image input controller 42 temporarily stores fluorescence image signals with respect to each frame outputted from the imaging control unit 27. Then, the ordinary image signals stored in the ordinary image input controller 41 and the fluorescence image signals stored in the fluorescence image input controller 42 are stored in the memory 44 via the bus.

The image processing unit 43 receives ordinary image signals and fluorescence image signal with respect to each frame read out from the memory 44, performs predetermined processing on these image signals, and outputs the resultant image signals to the bus.

The video output unit 45 receives an ordinary image signal and a fluorescence image signal outputted from image processing unit 43 via the bus, generates a display control signal by performing predetermine processing on the received signals, and outputs the display control signal to monitor 4.

The operating section 46 receives operator inputs, such as various types of operational instructions and control parameters. The TG 47 outputs drive pulse signals for driving the high sensitivity image sensor 24 and image sensor 26 of camera head section 20, and LD drivers 51, 54 of the light source apparatus 2, to be described later. The CPU 48 performs overall control of the system.

As described above, the signal cable 5c is connected to the processor 3. The signal cable 5c includes a signal line for transmitting an image signal outputted from the imaging control unit 27 of the camera head section 20, a control line for transmitting a control signal outputted from the processor 3, and the like.

As illustrated in FIG. 8, the light source apparatus 2 includes an excitation LD light source 50 that emits a 445 nm blue light, a LD driver 51 for driving the excitation light source 50, and a condenser lens 52 that condenses the blue light emitted from the excitation light source 50 and inputs the condensed light to the excitation light optical cable 5a of the cable 5.

The light source apparatus 2 further includes a near infrared LD light source 53 that emits a 750 to 790 nm near infrared light, a LD driver 54 for driving the near infrared LD light source 53, and a condenser lens 55 that condenses the near infrared light emitted from the near infrared LD light source 53 and inputs the condensed light to the near infrared light optical cable 5b of the cable 5.

The excitation light optical cable 5a includes two fiber bundles and the excitation light guided by each fiber bundle is inputted to each corresponding white light output section 28 of the camera head section 20. The near infrared light optical cable 5b also includes two fiber bundles and the near infrared light guided by each fiber bundle is inputted to each corresponding near infrared light output section 29 of the camera head section 20.

In the present embodiment, the near infrared light is used as the light other than the white light for capturing an ordinary image, but the light other than the white light is not limited to a light in the near infrared wavelength range, and a light determined, as appropriate, according to the type of fluorescent pigment used or the type of living tissue for causing autofluorescence may be used.

An operation of the rigid scope system of the present embodiment will now be described.

First, the excitation light optical cable 5a and near infrared light optical cable 5b of the cable 5 are connected to the light source apparatus 2, and the signal cable 5c of the cable 5 is connected to the processor 3. If the camera head section 20 and cable 5 are structured to be detachably attachable, the connector C of the cable 5 is connected to the camera head section 20. Further, the rigid scope body 30 is connected to the camera head section 20 to which the cable 5 has been connected in the manner described above. Note that the rigid scope body 30 is connected to the camera head section 20 such that the optical axes of the white light output sections 28 and near infrared light output sections 29 in the camera head section 20 and optical axes of the fiber bundles 30e, 30f in the rigid scope body 30 are placed adjacent to each other. When the camera head section 20 and rigid scope body 30 are connected, the light blocking section 6 provided on the connection section 20a of the camera head section 20 and the light blocking section 30h provided on the connection section 30a of the rigid scope body 30 are brought into close contact with each other, whereby the relay lens 30g in the rigid scope body 30 and imaging unit 35 in the camera head section 20 are almost entirely protected from the light.

Then, the user inputs an instruction to emit the white light and near infrared light using the operating section 46 of the processor 3 or the operation button 20b of the camera head section 20, and emissions of the excitation light and near infrared light are started from the excitation light source 50 and the near infrared LD light source 53 respectively in response to the emission start instruction.

The excitation light L5 emitted from the excitation light source 50 and the near infrared light L2 emitted from the near infrared light source 53 of the light source apparatus 2 are guided by the cable 5 and inputted to the white light output sections 28 and near infrared light output sections 29 respectively.

The excitation light inputted to the white light output sections 28 is guided by the multimode optical fibers 28a in the white light output sections 28 and projected on the phosphors 28b. Then, white light emitted from the phosphors 28b illuminated by the excitation light is outputted toward fiber bundles 30e, 30f of the rigid scope body 30 via the light projection windows 28c.

In the mean time, the near infrared light inputted to the near infrared light output sections 29 is guided by the multimode optical fibers 29a in the near infrared light output sections 29, inputted to the light projection windows 29b, and outputted toward the fiber bundles 30e, 30f of the rigid scope body 30 via the light projection windows 29b.

Then, the white light and near infrared light outputted from the white light output sections 28 and near infrared light output sections 29 are mixed and inputted to the fiber bundles 30e, 30f of the rigid scope body 30.

The white light L1 and near infrared light L2 guided by the fiber bundles 30e, 30f in the rigid scope body 30 are projected on the examination area from the tip of the rigid scope body 30.

Then, an ordinary image, which is based on a reflection light reflected from the examination area by the projection of the white light L1, and a fluorescence image, which is based on fluorescence emitted from the examination area by the projection of the near infrdred light L2, are captured. Note that ICG is administered to the examination area in advance and fluorescence emitted from the ICG is captured.

More specifically, when capturing the ordinary image, an ordinary image L3, which is based on the reflection light reflected from the examination area by the projection of the white light L1, is inputted from a tip portion of the insertion member 30b, guided by the relay lens 30g in the insertion member 30b, and outputted to the imaging unit 35 in the camera head section 20.

The ordinary image L3 inputted to the imaging unit 35 is reflected in a right angle direction toward the image sensor 26 by the dichroic prism 21, formed on the imaging surface of the image sensor 26 by the second image forming optical system 25, and sequentially captured by the image sensor 26 at a predetermined frame rate.

Each ordinary image signal sequentially outputted from the image sensor 26 is subjected to CDS/AGC (correlated double sampling/automatic gain control) and A/D conversion in the imaging control unit 27 and sequentially outputted to the processor 3 through the signal cable 5c.

Then, each ordinary image signal inputted to the processor 3 is temporarily stored in the ordinary image input controller 41 and then stored in the memory 44. The ordinary image signals read out from the memory 44 with respect to each frame are subjected to tone correction and sharpness correction in the image processing unit 43, and sequentially outputted to the video output unit 45.

The video output unit 45 generates a display control signal by performing predetermined processing on the inputted ordinary image signals and sequentially outputs display control signals to the monitor 4 with respect to each frame. The monitor 4 displays an ordinary image based on the inputted display control signals.

In the mean time, when capturing the fluorescence image, a fluorescence image L4, which is based on fluorescence emitted from the examination area by the projection of the near infrared light L2 is inputted from the tip portion of the insertion member 30b, guided by the relay lens 30g in the insertion member 30b, and outputted to the imaging unit 35 of the camera head section 20.

The fluorescence image L4 inputted to the imaging unit 35 is transmitted through the dichroic prism 21 and excitation light cut filter 22, then formed on the imaging surface of the high sensitivity image sensor 24 by the first image forming optical system 23, and sequentially captured by the high sensitivity image sensor 24 at a predetermined frame rate.

Each fluorescence image signal sequentially outputted from the high sensitivity image sensor 24 is subjected to CDS/AGC (correlated double sampling/automatic gain control) and A/D conversion in the imaging control unit 27 and sequentially outputted to the processor 3 through the signal cable 5c.

Then, each fluorescence image signal inputted to the processor 3 is temporarily stored in the fluorescence image input controller 42 and then stored in the memory 44. The fluorescence image signals read out from the memory 44 with respect to each frame are subjected to predetermined processing in the image processing unit 43, and sequentially outputted to the video output unit 45.

The video output unit 45 generates a display control signal by performing predetermined processing on the inputted fluorescence image signals and sequentially outputs display control signals to the monitor 4 with respect to each frame. The monitor 4 displays a fluorescence image based on the inputted display control signals.

According to the rigid scope system of the embodiment described above, the white light output sections 28 and near infrared light output sections 29 are provided in the camera head section 20, and the white light and near infrared light outputted from these sections are received and guided by the rigid scope body 30, whereby the white light and near infrared light are projected on the examination area. This eliminates the need to connect a light guide to the rigid scope body 30, as required in the conventional rigid scope apparatus, resulting in an overall downsizing of the apparatus and operability improvement by saving the effort of connecting and maneuvering the light guide.

In the rigid scope system 1 of the embodiment described above, the emission of the white light and near infrared light is started when an instruction to start emission of the white light and infrared light is given via the operating section 46 or operation button 20b. There may be a case in which the emission start instruction is given when the camera head section 20 is not accidentally connected to the rigid scope body 30 and in such a case it is necessary to prevent the near infrared laser light outputted from the near infrared light output sections 29 and blue laser light outputted from the white light output sections 28 of the camera head section 20 from being accidentally incident on the human eyes. Similarly, in the case where the emission start instruction is given when the camera head section 20 is not connected to the light source apparatus 2 via the cable 5, it is necessary to prevent the blue laser light and near infrared laser light from being accidentally incident on the human eyes.

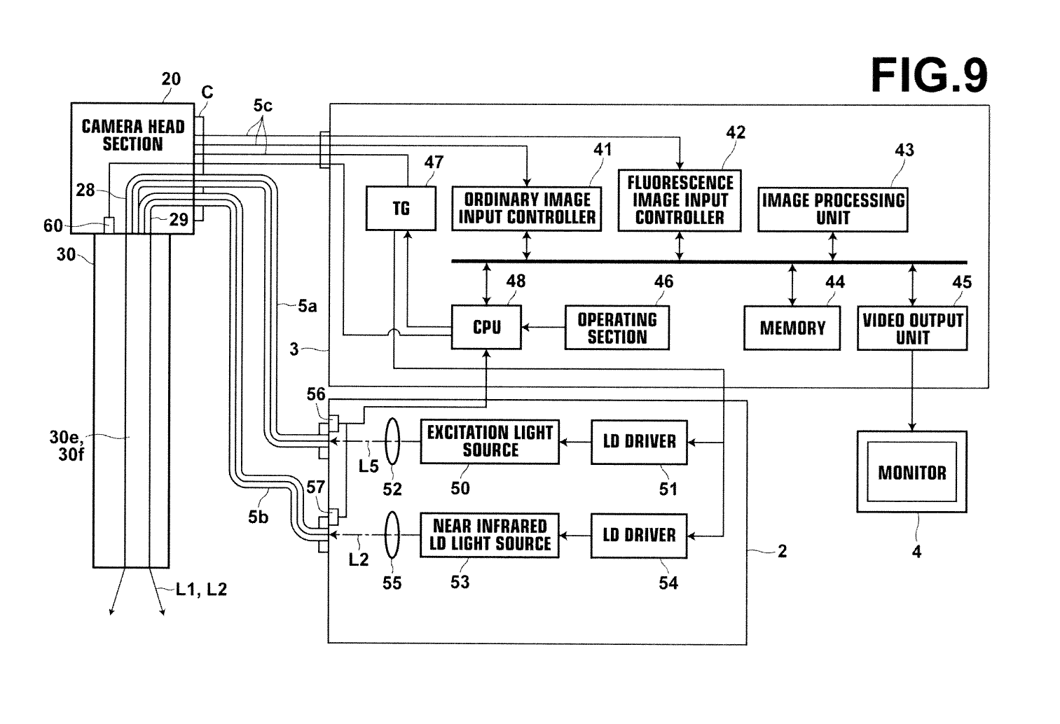

Consequently, as illustrated in FIG. 9, a rigid scope connection detection section 60 may be provided to detect whether or not the camera head section 20 and rigid scope body 30 are connected to each other, as well as providing camera head connection detection sections 56, 57 to detect whether or not the light source apparatus 2 and camera head section 20 are connected to each other. As for the rigid scope connection detection section 60 and camera head connection detection sections 56, 57, for example, optical sensors, other sensors, electrical switches, or the like may be used. The camera head connection detection sections 56, 57 shown in FIG. 9 are detectors that detect whether or not the camera head section 20 is connected to the light source apparatus 2 via the cable 5 by detecting whether or not the cable 5 is connected to the light source apparatus 2. In the case where the connector C of the cable 5 is structured to be detachably attachable to the camera head section 20, detection as to whether or not the connector C of the cable 5 is connected to the camera head section 20 may be performed. Otherwise, detection of both the connection between the cable 5 and light source apparatus 2, and the connection between the cable 5 and camera head section 20 may be performed.

Then, in the case where the connection between the rigid scope body 30 and camera head section 20 is detected by the rigid scope connection detection section 60 and the connection between the camera head section 20 and the light source apparatus 2 is detected by the camera head connection detection sections 56, 57, the CPU 48 of the processor 3 may cause the excitation light source 50 and near infrared LD light source 53 to emit the excitation light and near infrared light respectively as the normal procedure, while if the connection between the rigid scope body 30 and camera head section 20 or the connection between the camera head section 20 and light source apparatus 2 is not detected, the CPU 48 may cause the excitation light source 50 and near infrared LD light source 53 not to emit the excitation light and near infrared light. The method for stopping the output of the laser light from the camera head section 20 or light source apparatus 2 is not limited to control the light source itself and the output of the laser light may be controlled by providing a shutter or the like.

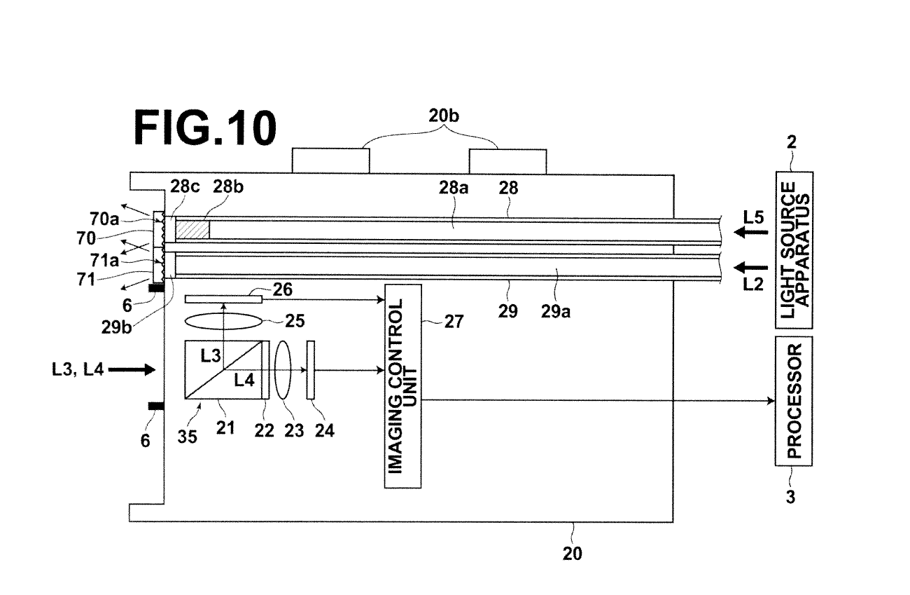

As illustrated in FIG. 10, a diffusion section 70 may be provided at the light projection window 28c of each white light output section 28 to diffuse the white light and a diffusion section 71 may be provided at the light projection window 29b of each near infrared light output section 29 to diffuse the near infrared light as a safety measure against laser output from the camera head section 20 in the case where the rigid scope body 30 is not connected to the camera head section 20. As for the diffusion sections 70, 71, for example, light shaping diffusers, diffusion films, diffusion filters, or the like may be used, that is, anything may be used as long as it is capable of diffusing and transmitting inputted light.

For example, in the case where the light shaping diffusers are used as the diffusion sections 70, 71, it is preferable that the diffusers are provided such that diffusion surfaces 70a, 71a on which a surface relief hologram pattern is formed becomes input face of the white light and near infrared light, i.e., the diffusion surfaces 70a, 71a are on the light output end face side of the camera head section 20, as illustrated in FIG. 10. Disposition of the diffusion surfaces 70a, 71a in the orientation described above may prevent degradation in diffusion function by the adherence of dirt or water to the diffusion surfaces.

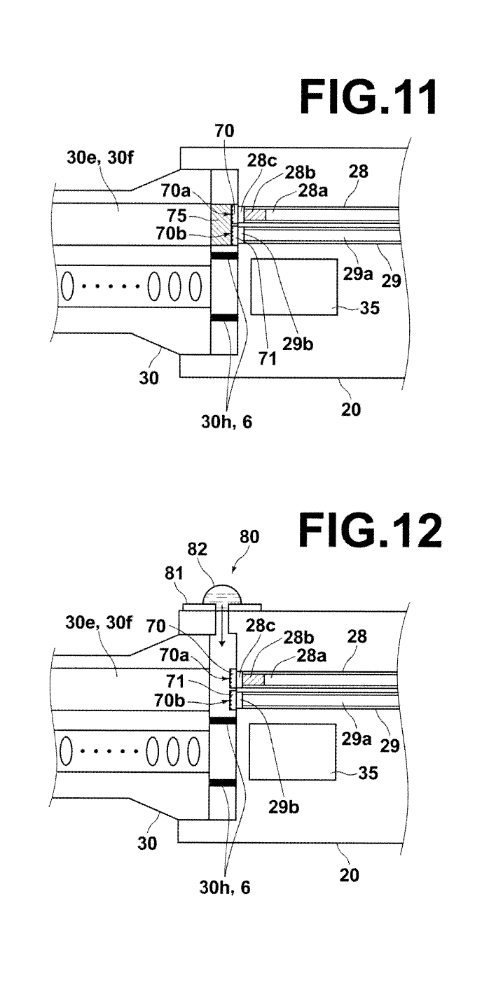

In the case where the diffusion sections 70, 71 are provided in the manner described above, a transmission member 75 having a refractive index identical to that of the light projection window 28c and light projection window 29b and/or that of the fiber bundles 30e, 30f and transmitting white light and near infrared light outputted from the diffusion sections 70, 71 may be provided between the diffusion sections 70, 71 and input faces of the fiber bundles 30e, 30f, as illustrated in FIG. 11. Provision of the transmission member 75 allows the diffusion sections 70, 71 of the camera head section 20 to be optically connected to the fiber bundles 30e, 30f of the rigid scope body 30 when the camera head section 20 is connected to the rigid scope body 30, whereby guiding efficiency of the white light and near infrared light may be improved.

As for the transmission member 75, a member made of an elastic material, such as a resin, is preferably used. Specific materials of the transmission member include, for example, LED silicone materials (SLJ9101, SLJ9102, SLJ9105, SLJ9106 (available from Asahi Kasei Corporation)) and high transparent urethane rubbers.

Use of such an elastic member as the transmission member 75 may improve the adhesion between the diffusion surfaces 70a, 71a of the diffusion sections 70, 71 and transmission member 75, and adhesion between the transmission member 75 and input faces of the fiber bundles 30e, 30f.

In particular, in the case where the diffusion sections 70, 71 are disposed such that the diffusion surfaces 70a, 71a are oriented to the output face side of the white light and near infrared light (input face side of the fiber bundles 30e, 30f of the rigid scope body 30), as illustrated in FIG. 11, diffusion effect of the diffusion surfaces 70a, 71a may be suppressed by improving the adhesion between the diffusion surfaces 70a, 71a of the diffusion section 70, 71 and transmission member 75, so that the guiding efficiency of the white light and near infrared light may further be improved.

The transmission member 75 may be provided on the side of the rigid scope body 30 or on the side of the camera head section 20 and preferably detachably attachable thereto. When foreign particles are adhered to the transmission member, such structure allows the transmission member to be detached to remove the foreign particles and reattached. The structure that allows the transmission member to be detachably attachable may be, for example, a structure in which the transmission member 75 is freely detached by hand from the rigid scope body 30 or camera head section 20 without being fixed thereto, or a structure in which the transmission member 75 is provided on a member which is detachably attachable to the rigid scope body 30 or camera head section 20.

The transmission member 75 may be provided regardless of whether or not the diffusion sections 70, 71 are provided, and provision of the transmission member 75 may prevent degradation in light guiding efficiency that may occur in a spatial optical connection.

Further, as illustrated in FIG. 12, a supply member 80 for supplying a liquid or gel having a refractive index identical to that of the light projection window 28c and light projection window 29b and/or that of the fiber bundles 30e, 30f to the camera head section 20 may be provided between the light output faces of the white light output sections 28 and near infrared light output sections 29 and input faces of the fiber bundles 30e, 30f of the rigid scope body 30.

The supply member 80 includes a storage section 82 in which the liquid or gel is stored and an installation member 81 having the storage section 82 thereon and is installed on the camera head section 20. Preferably, the installation member 81 is structured to be detachably attachable to the camera head section 20, i.e., structured to be disposable. The storage section 82 is made of a material deformable when pressed by a human hand, such as a resin, and the liquid or gel filled therein has a refractive index identical to that of the light projection window 28c and light projection window 29b. Such liquids may include matching oils and matching gels. For example, the refractive liquid available from Cargille Inc., describe in http://www.cargille.com/refractivesstandards.shtml may be used.

After the camera head section 20 is connected to the rigid scope body 30, the liquid or the like in the storage section 82 is discharged via a hole formed through the installation member and in the camera head section 20 by the deformation of the storage section 82 pressed by a human hand.

The discharged liquid or the like is supplied between the diffusion sections 70, 71 provided on the camera head section 20 and input faces of fiber bundles 30e, 30f of the rigid scope body 30, which causes the diffusion sections 70, 71 and fiber bundles 30e, 30f to be optically connected, whereby light guiding efficiency may be improved.

In the case where the supply member 80 described above is provided, the diffusion sections 70, 71 may be provided such that the diffusion surfaces 70a, 71a are oriented to the side of the input face of the fiber bundles 30e, 30f, as illustrated in FIG. 11. Such structure allows diffusion effect of the diffusion surfaces 70a, 71a of the diffusion sections 70, 71 to be suppressed by contacting the diffusion surfaces 70a, 71a with the liquid or the like supplied from the supply member 80, so that the guiding efficiency of the white light and near infrared light may be improved.

But the orientation of the diffusion surfaces 70a, 71a is not limited to this, and they may be oriented to the opposite direction, i.e., to the side of the light output faces of the white light output sections 28 and near infrared light output sections 29. Provision of the diffusion sections 70, 71 in the manner described above may prevent degradation in diffusion effect of the diffusion sections 70, 71 due to adherence of the liquid or the like supplied from the supply member 80 to the diffusion sections 70, 71. Further, the supply member 80 may be provided in the case where the diffusion sections 70, 71 are not provided, and provision of the supply member 80 may improve guiding efficiency of the white light and near infrared light.

Here, the liquid or gel having a refractive index equivalent to that of the light projection window 28c of the white light output section 28 and the light projection window 29b of the near infrared light output section 29 is supplied. But, in the case where the white light output section 28 and near infrared output section 29 are formed of one multimode optical fiber, a liquid or gel having a refractive index identical to that of the multimode optical fiber may be supplied. In short, all that is required is to supply a liquid or gel having a refractive index identical to that of the material adjacent to the output faces of the white light output section 28 and near infrared light output section 29.

The supply member 80 may be provided regardless of whether the diffusion sections 70, 71 are provided, and provision of the supply member 80 may prevent degradation in light guiding efficiency that may occur in a spatial optical connection.

In the rigid scope system 1 of the embodiment described above, the white light outputted from the white light output section 28 and near infrared light outputted from the near infrared light output section 29 are mixed and inputted to the fiber bundles 30e, 30f of the rigid scope body 20. But, the invention is not limited to such structure and, for example, as the fiber bundles provided in the rigid scope body 20, a white light fiber bundle for guiding the white light and a near infrared light fiber bundle for guiding the near infrared light may be provided separately, and the white light outputted from the white light output section 28 may be inputted to the white light fiber bundle while the near infrared light outputted from the near infrared light output section 29 may be inputted to the near infrared light fiber bundle. Such structure allows selection of fiber bundles most appropriate for the wavelengths of the light to be guided, whereby the transmittance of the white light and near infrared light may be improved. Note that light receiving portions of the white light fiber bundle and near infrared fiber bundle correspond to the specific wavelength light receiving sections in the claims.

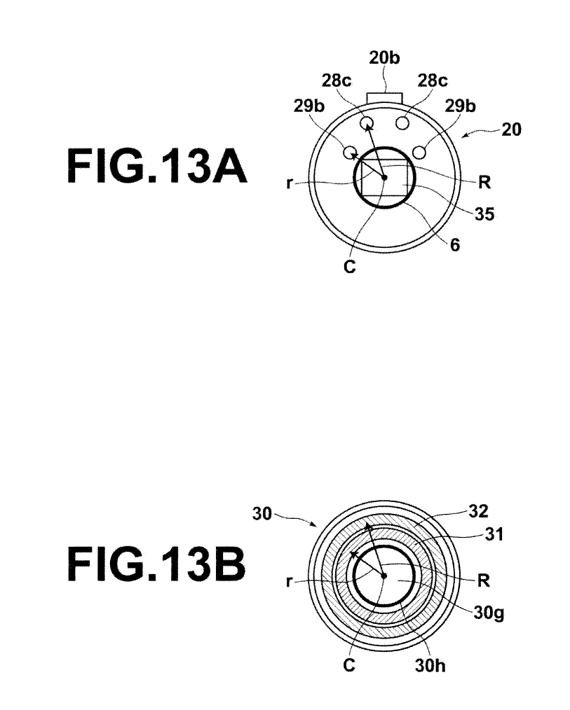

As for the structure in which the near infrared light fiber bundle and white light fiber bundle are provided separately as described above, for example, the light projection window 28c of the white light output section 28 in the camera head section 20 may be provided at a position with a distance “R” from the center C of the cross-section (connection face) of the camera head section 20 while the light projection window 29b of the near infrared light output section 29 may be provided at a position with a distance “r”, which is different from the distance “R”, from the center C of the cross-section (connection face) of the camera head section 20, as illustrated in FIG. 13A. To this, in the rigid scope body 30, a white light fiber bundle 32 may be disposed at a position with the distance “R” from the center C of the cross-section (connection face) of the rigid scope body 30 while a near infrared fiber bundle 31 may be disposed at a position with the distance “r” from the center C of the cross-section (connection face) of the rigid scope body 30, as illustrated in FIG. 13B. Note that the center C of the cross-section (connection face) of the camera head section 20 and the center C of the cross-section (connection face) of the rigid scope body 30 are disposed on the same central axis when the camera head section 20 is connected to the rigid scope body 30.

Then, if the white light fiber bundle 32 and near infrared fiber bundle 31 are concentrically formed with respect to the cross-sectional center C, as illustrated in FIG. 13B, the white light outputted from the white light output sections 28 may be inputted to the white light fiber bundle 32 while the near infrared light outputted from the near infrared light output sections 29 may be inputted to the near infrared light fiber bundle 31 even when the camera head section 20 rotates in a circumferential direction relative to the rigid scope body 30. Thus, the white light and near infrared light are never mixed and inputted to each fiber bundle. Note that the white light fiber bundle 32 and near infrared fiber bundle 31 do not necessarily have a circular shape and may have an arc shape, such as a semicircular shape or a crescent shape.

In the rigid scope system 1 of the embodiment described above, the white light output section 28 is formed of the excitation light multimode optical fiber 28a, phosphor 28b, and light projection window 28c, but the structure of the white light output section 28 is not limited to this, and the white light output section 28 may be formed, for example, of a white light source, such as xenon lamp or halogen lamp, provided in the light source apparatus 2 and a white light multimode optical fiber for guiding the white light emitted from the white light source and inputted thereto via the cable 5. Alternatively, a LED (light emitting diode) that emits the white light may be provided in the camera head section 20 as the white light output section 28.

Further, for the near infrared light output section 29 also, a LED or LD (laser diode) that emits the near infrared light may be provided in the camera head section 20.

1. A rigid scope apparatus, comprising:
a rigid scope body to be inserted into a body for guiding and projecting an inputted illumination light on an examination area of the body, and receiving and guiding a light from the examination area caused by the projection of the illumination light; and
a camera head section to be connected to the rigid scope body and having an imaging section for receiving the light from the examination area and guided by the rigid scope body, and outputting an image signal, wherein:
the camera head section includes an illumination light output section for outputting the illumination light; and
the rigid scope body receives and guides the illumination light outputted from the illumination light output section.

2. The rigid scope apparatus of claim 1, wherein the illumination light output section includes an optical fiber for guiding and outputting the illumination light, or a phosphor that emits the illumination light and an optical fiber for guiding an excitation light to be projected on the phosphor.

3. The rigid scope apparatus of claim 1, wherein:
the apparatus includes a rigid scope connection detection section for detecting as to whether or not the rigid scope body is connected to the camera head section; and
the illumination light output section is a section that outputs the illumination light while connection between the rigid scope body and the camera head section is detected by the rigid scope connection detection section and does not output the illumination light while connection between the rigid scope body and the camera head section is not detected.

4. The rigid scope apparatus of claim 3, wherein:
the apparatus includes a camera head connection detection section for detecting as to whether or not the camera head section is connected to a light source apparatus that emits a light to be inputted to the camera head section; and
the illumination light output section is a section that outputs the illumination light while connection between the rigid scope body and the camera head section is detected by the rigid scope connection detection section and connection between the camera head section and the light source apparatus is detected by the camera head connection detection section, and does not output the illumination light while connection between the rigid scope body and the camera head section or connection between the camera head section and the light source apparatus is not detected.

5. The rigid scope apparatus of claim 1, wherein at least one of the rigid scope body and the camera head section includes a light blocking section for preventing the illumination light outputted from the illumination light output section from entering into the imaging section of the camera head section.

6. The rigid scope apparatus of claim 1, wherein the illumination light output section comprises a section that outputs a plurality of illumination lights having different wavelength ranges.

7. The rigid scope apparatus of claim 6, wherein one of the plurality of illumination lights comprises a white light.

8. The rigid scope apparatus of claim 6, wherein one of the plurality of illumination lights comprises a near infrared light or an autofluorescence excitation light for exciting autofluorescence in the examination area.

9. The rigid scope apparatus of claim 6, wherein the rigid scope body includes a light receiving section for receiving a light having the plurality of illumination lights mixed therein.

10. The rigid scope apparatus of claim 6, wherein the rigid scope body includes a plurality of specific wavelength light receiving sections for receiving the plurality of illumination lights separately.

11. The rigid scope apparatus of claim 10, wherein each of the plurality of specific wavelength light receiving sections is provided at a different radial position from a center position of a connection face of the rigid scope body with the camera head section.

12. The rigid scope apparatus of claim 11, wherein the plurality of specific wavelength light receiving sections is provided concentrically with respect to the center position.

13. The rigid scope apparatus of claim 1, wherein the illumination light output section includes a diffusion section for diffusing the illumination light.

14. The rigid scope apparatus of claim 1, wherein the apparatus includes a transmission member between an output face of the illumination light output section and a light receiving face of the rigid scope body for receiving the illumination light, the transmission member having a refractive index substantially identical to a refractive index adjacent to the output face or a refractive index adjacent to the light receiving face, and transmitting the illumination light outputted from the illumination light output section.

15. The rigid scope apparatus of claim 14, wherein the transmission member comprises a member having elasticity.

16. The rigid scope apparatus of claim 14, wherein the transmission member comprises a member formed of a resin.

17. The rigid scope apparatus of claim 14, wherein the transmission member is detachably attachable to the camera head section or the rigid scope body.

18. The rigid scope apparatus of claim 1, wherein the apparatus includes a supply member for supplying a liquid or gel between an output face of the illumination light output section for outputting the illumination light and a light receiving face of the rigid scope body for receiving the illumination light, the liquid or gel having a refractive index substantially identical to a refractive index adjacent to the output face or a refractive index adjacent to the light receiving face.

19. The rigid scope apparatus of claim 1, wherein the illumination light output section is provided in a peripheral portion of the imaging section.

20. The rigid scope apparatus of claim 15, wherein the transmission member comprises a member formed of a resin.
